(19) United States
(12) Patent Application Publication
(10) Pub. No.: US 2022/0168165 A1
(43) Pub. Date: Jun. 2, 2022

(54) Wheeled Apparatus For Transporting A Portable Oxygen Concentrator Or Other Medical Device

(71) Applicant: Heather Farrell, Yokohama (JP)

(72) Inventor: Heather Farrell, Yokohama (JP)

(21) Appl. No.: 17/106,208

(22) Filed: Nov. 30, 2020

(51) Int. Cl. A61G 12/00

(52) U.S. Cl. CPC A61G 12/008; A61G 2203/10

(57) ABSTRACT

A wheeled apparatus is provided that is intended to be used for the safe movement of various medical devices and/or portable oxygen concentrators (POCs) that are now available on the market or through private or public health care systems, or will become available on the market or through private or public health care systems as medical device technology continues to progress. The dimensions for the present invention are variable to suit the specific medical device that the wheeled apparatus will be transporting. The wheeled apparatus additionally provides a highly maneuverable mobility device that can assist a user to have improved stability and/or balance while walking. The wheeled apparatus may be leaned on by a user and thereby used for support while transporting vital oxygen therapy equipment that is absolutely essential for the user. The wheeled apparatus may include a handle assembly having a telescopic handle, one or more sets of wheels or independently rotatable wheels, and a case body having mesh panels to provide ventilation and a plurality of pass through openings for receiving one or more connective apparatus.

### CROSS-REFERENCES TO RELATED APPLICATIONS

This non-provisional utility application takes priority to the previously filed provisional application: Application No. 62/894,845 by inventor Heather Farrell, which is hereby incorporated in its entirety by reference.

### BRIEF DESCRIPTION OF THE INVENTION

The present invention is generally related to apparatus for transporting medical devices, and more particularly related to a wheeled apparatus specifically designed for transporting a portable oxygen concentrator or other medical device.

The herein disclosed wheeled apparatus is intended to be used for the safe movement of various medical devices and/or portable oxygen concentrators that are now available on the market or through private or public health care systems, or will become available on the market or through private or public health care systems as medical device technology continues to progress. As such, the dimensions for the present invention are variable to suit the specific medical device that the wheeled apparatus will be transporting.

A further benefit of the herein disclosed wheeled apparatus is to provide a highly maneuverable mobility device that can additionally assist a user to have improved stability and/or balance while walking. The wheeled apparatus may be leaned on by a user and thereby used for support while transporting vital oxygen therapy equipment that is absolutely essential for the user.

The wheeled apparatus for transporting a portable oxygen concentrator or other medical device may include a handle assembly having a telescopic handle, one or more sets of wheels or independently rotatable wheels, and a case body having mesh panels to provide ventilation for the portable oxygen concentrator or other medical device and a plurality of pass through openings for receiving one or more connective apparatus for the portable oxygen concentrator or other medical device.

### BACKGROUND OF THE INVENTION

A portable oxygen concentrator (commonly referred to as a “POC” or plurality of “POCs”) is a medical device that provides oxygen therapy to patients that require greater oxygen concentrations than the levels ordinarily present in ambient air.

While POCs are designed to be small enough to be portable, a POC generally weights about 10 pounds and therefore poses a significant weight for a patient to carry. For this reason, POCs are generally transported in a backpack or handheld bag. These POC-containing backpacks or handheld bags are heavy enough that patients, who are usually weak and fatigued, cannot carry the POC-containing backpack or handheld bag themselves and must therefore rely on a caregiver to transport the POC-containing backpack or handheld bag. it is apparent that these backpacks and handheld bags are not meeting the needs of patients who require the use of a POC to provide concentrated oxygen.

It would therefore be advantageous to provide a wheeled apparatus for transporting a POC, The herein disclosed wheeled apparatus for transporting a portable oxygen concentrator provides a purpose built bag (or case) for carrying a POC that enables easy movement of the medical device in all directions without the user having to tilt the bag or directly bear the weight of the POC device itself.

### BRIEF DESCRIPTION OF THE SEVERAL VIEWS OF THE DRAWING

FIG. 1 illustrates a front view of the herein disclosed wheeled apparatus for transporting a portable oxygen concentrator or other medical device, in accordance with an exemplary embodiment of the present invention;

FIG. 2 illustrates a first side view of the herein disclosed wheeled apparatus for transporting a portable oxygen concentrator or other medical device, in accordance with an exemplary embodiment of the present invention;

FIG. 3 illustrates a second side view (of the side opposite the view illustrated in FIG. 2) of the herein disclosed wheeled apparatus for transporting a portable oxygen concentrator or other medical device, in accordance with an exemplary embodiment of the present invention;

FIG. 4 illustrates a rear view of the herein disclosed wheeled apparatus for transporting a portable oxygen concentrator or other medical device, in accordance with an exemplary embodiment of the present invention;

FIG. 5 illustrates a bottom view of the herein disclosed wheeled apparatus for transporting a portable oxygen concentrator or other medical device, in accordance with an exemplary embodiment of the present invention; and

FIG. 6 illustrates an isometric view of an ideal embodiment of the herein disclosed wheeled apparatus for transporting a portable oxygen concentrator or other medical device, in accordance with an exemplary embodiment of the present invention.

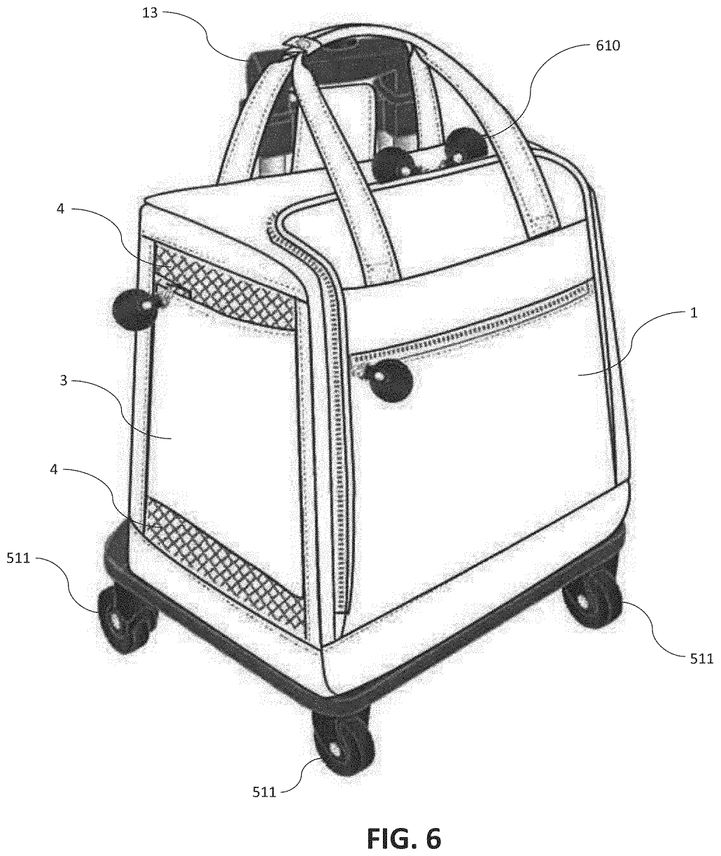

### DETAILED DESCRIPTION OF THE INVENTION

The present invention is generally related to apparatus for transporting medical devices, and more particularly related to a wheeled apparatus specifically designed for transporting a portable oxygen concentrator or other medical device. The herein disclosed wheeled apparatus is intended to be used for the safe movement of various medical devices, oxygen tanks/cylinders, and/or portable oxygen concentrators that are now available on the market or through private or public health care systems, or will become available on the market or through private or public health care systems as medical device technology continues to progress. To accommodate the wide variety of medical devices that are now available or will become available, the herein disclosed wheeled apparatus may be formed or manufactured in a wide variety of sizes and/or configurations while continuing to embody the present invention and all such sizes and/or configurations are intended to be included in this specification.

An exemplary embodiment of the herein disclosed wheeled apparatus for transporting a portable oxygen concentrator or other medical device includes: a case body having a plurality of mesh panels to provide ventilation, wherein the case body is sized to receive the portable oxygen concentrator or other medical device, the plurality of mesh panels are positioned to provide ventilation to the portable oxygen concentrator or other medical device, and wherein the case body forms a plurality of pass through openings for receiving one or more connective apparatus for the portable oxygen concentrator or other medical device; one or more sets of wheels for supporting the case body and the portable oxygen concentrator or other medical device and for facilitating manual rolling of the case body and the portable oxygen concentrator or other medical device; and a handle assembly that is retractable and telescopic.

The herein disclosed wheeled apparatus may be advantageously utilized to transport any type of medical device and is preferably utilized to transport a portable oxygen concentrator. A portable oxygen concentrator may be referred to as a “POC” and is a particular type of medical device designed for use by patients that require greater oxygen concentrations than the levels present in ambient air. While such patients are currently advised or prescribed to use a POC, in the future such patients may be advised or prescribed to use an alternative medical device that operates to provide improved oxygen intake or, more generally, to provide improved breathing and all such alternative medical devices are intended to be included herein as medical devices that may be advantageously transported via the present invention. The medical device that is transported by the present invention will mostly be referred to as a POC or a portable oxygen concentrator, but may alternatively be referred to as an oxygen producing device, oxygen producing equipment, a device for oxygen therapy, equipment for oxygen therapy, supplementary oxygen, oxygen therapy accessories, oxygen tank(s), oxygen cylinder(s), or generally as a medical device.

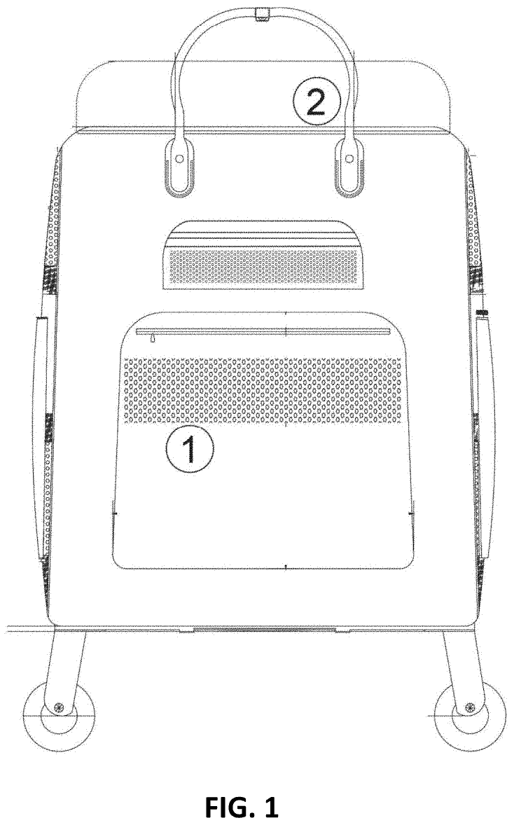

Referring to FIG. 1, a front view of the herein disclosed wheeled apparatus for transporting a portable oxygen concentrator or other medical device is shown. The wheeled apparatus includes a case body forming front pocket 1 and which may include an expandable case body top 2. Expandable case body top 2 may have a long and flat zipper located in the top center which opens directly to the interior of the wheeled apparatus to provide further access, venting, and/or pass through options directly to the interior. The purpose of expandable case body top 2 is to provide an adjustable size for the wheeled apparatus that may accommodate a wide variety of different sized POC units or other medical device units that are currently available or may become available in the future.

Front pocket 1 may be closeable via a fastener, such as a horizontally oriented zipper for example, located at the top of front pocket 1. The horizontally oriented zipper may be a double zipper-pull zipper that facilitates the ad hock creation of an additional pass through opening at front pocket 1 that opens through to an interior of the wheeled apparatus for transporting a portable oxygen concentrator or other medical device; all zippers included in the present invention may optionally be double zipper-pull zippers to facilitate the ad hock creation of additional pass through openings. Front pocket 1 may form a ventilation panel; the ventilation panel may be formed by a mesh material used to form a part, or all, of front pocket 1.

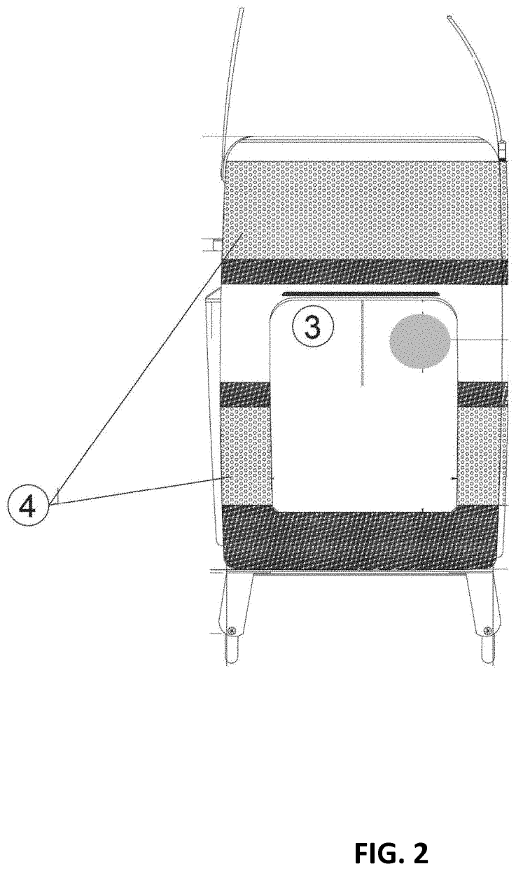

Referring to FIG. 2, a first side view (which may be a right side view) of the herein disclosed wheeled apparatus for transporting a portable oxygen concentrator or other medical device is shown. The case body forms first side pocket 3 and one or more first side body ventilation panels 4. First side pocket 3 may he closeable via a fastener, such as a zipper or a zipper having a duel or double zipper-pull that extends along three sides of first side pocket 3 for example. First side pocket 3 may additionally form first pass through opening 3b, which may be a hole, a flap-covered opening, or any other aperture formed through first side pocket 3 and through the case body and into the interior of the wheeled apparatus. One or more first side body ventilation panels 4 may be formed by utilizing a mesh or otherwise porous material to form one or more side panels of the case body, and are intended to provide targeted ventilation to the POC or other type of medical device stored in the interior of the wheeled apparatus.

It should be noted that the purpose of all pass through openings (such as first pass through opening 3b, second pass through opening 5b, first back pass through pocket 9, second back pass through pocket 10, and cavity zipper 610 on top flap of the wheeled apparatus), O-rings, and double zipper-pull zippers are to provide oxygen delivery hoses, battery cables, or other connective apparatus openings or apertures through which to pass from the interior of the wheeled apparatus to the exterior of the wheeled apparatus easily or comfortably enough that bending and/or kinking of the oxygen delivery hoses, battery cables, or other connective apparatus does not occur. One or more of the pass through openings may be rubber ear-phone holes (or headphone holes).

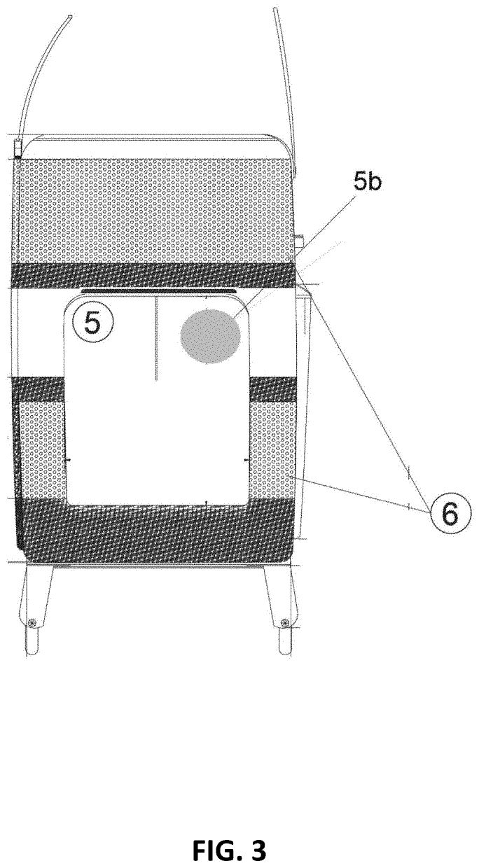

Referring to FIG. 3, a second side view (which may be a left side view) of the herein disclosed wheeled apparatus for transporting a portable oxygen concentrator or other medical device is shown. The case body forms second side pocket 5 and one or more second side body ventilation panels 6. Second side pocket 5 may be closeable via a fastener, such as a zipper or a zipper having a duel or double zipper-pull that extends along three sides of second side pocket 5 for example. Second Side pocket 5 may additionally form second pass through opening 5b, which may be a hole, a flap-covered opening, or any other aperture formed through second side pocket 5 and through the case body and into the interior of the wheeled apparatus. One or more second side body ventilation panels 6 may be formed by utilizing a mesh or otherwise porous material to form one or more side panels of the case body, and are intended to provide targeted ventilation to the POC or other type of medical device stored in the interior of the wheeled apparatus. The strategically positioned mesh panels are intended to provide targeted ventilation to a wide variety of POCs on the market today because the POCs available on the market today have intake and/or outtake vents in various locations on the physical devices. The various ventilation panels (on the front side, the first side, the second side, and the rear of the herein disclosed wheeled apparatus) along with the top zippered opening allow for targeted venting on all sides of a POC stored within to avoid the POC overheating within the wheeled apparatus.

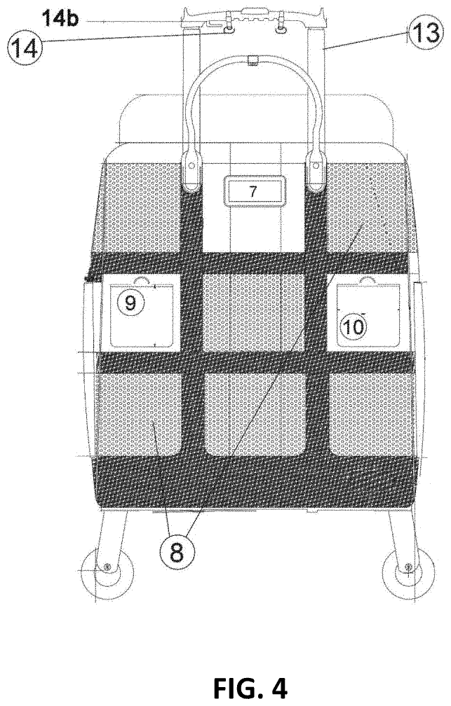

Referring to FIG. 4, a rear view of the herein disclosed wheeled apparatus for transporting a portable oxygen concentrator or other medical device is shown. The case body is at least partially formed of a porous material 8, such as a mesh material, to provide ventilation to the interior. The case body additionally forms first back pass through pocket 9 and second back pass through pocket 10, which may be positioned on opposite sides of the back of the case body and which are intended to receive one or more POC or other medical device connective apparatus in an anti-kink or kink-resistant manner, such as an oxygen hose or a power cable for example.

The wheeled apparatus for transporting a POC or other medical device may include a handle apparatus that has one or more hooks 14 for detachably connecting bags, purses, or connective apparatus. One or more hooks 14 may be utilized to organize connective apparatus; for example, an oxygen hose or a power cord may be temporarily placed through one or more of the hooks and thereby constrained from interfering with the patient's activities or thereby directed towards the desired target. The handle apparatus 13 may be telescopic so that the length is variable at the user's discretion and may include a braking mechanism that allows the user to apply a brake to the one or more sets of wheels via a finger-operated level located on handle apparatus 13. ID tag 7 for identifying the patient and/or the user may be formed on the back side of the case body.

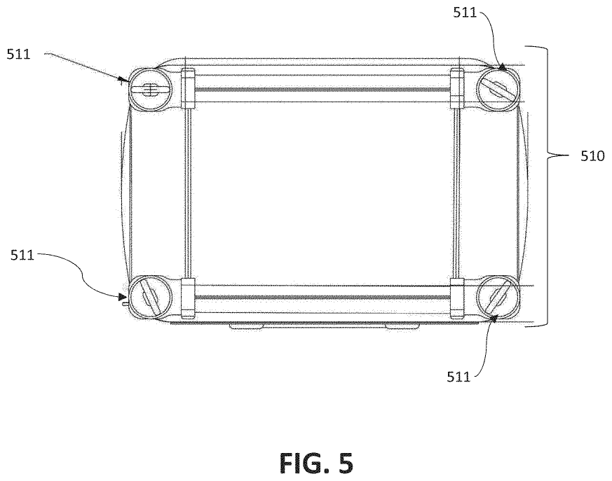

Referring to FIG. 5, a bottom view of the herein disclosed wheeled apparatus for transporting a portable oxygen concentrator or other medical device is shown. As can be seen in FIG. 5 and in the preceding figures, one or more sets of wheels 510, or a plurality of independently rotatable wheels 511, provide support to the case body and the medical device stored within. The one or more sets of wheels facilitate manual rolling of the case body and thus the medical device, and are able to provide a high degree of maneuverability and thus the overall device can provide a level of mobility assistance to a user or patient. For example, a user may be able to lean on or otherwise steady himself or herself while using handle assembly 13. And with the possible addition of a hand brake functionally embedded in the handle assembly, the user can control and/or stop the movement of the wheeled apparatus for any reason and at any time, thus increasing the user's sense of safety and independent mobility. In a preferred embodiment, the present invention includes four independently rotatable (or multi-directional spinning) wheels. The wheels may have a locking functionality. Alternatively, the wheels may be functionally connected to a brake mechanism of handle assembly 13.

Preferred embodiments of the present invention may additionally, or optionally, include one or more internal restraining straps to position and hold the POC or other medical device in a preferred location in the interior of the case body. Preferred embodiments may also include one or more accessory bags for storing medical equipment, an expandable zippered panel on one or both sides of the case body, and/or a motorized fan positioned on or within the case body to provide increased ventilation to the interior of the case body. In certain embodiments, it is possible to detach the case body from the one or more wheels (or from a frame supporting the one or more wheels) to facilitate carrying the case body like a backpack or a handbag.

It should be noted that an embodiment of the present invention may be improved through the addition of a capability to adjust, collapse, attach to, detach from, or convert in order to provide an adjustable size and/or dimension to facilitate use with various POCs and other medical devices. Alternatively, or additionally, one or more accessory bags may be utilized that may attach to the overall wheeled apparatus unit. Any advancements in storage technology that when applied to the herein disclosed wheeled apparatus are able to provide these capabilities are intended to be included herein.

All dimensions shown in the various figures are intended to be exemplary and are included to illustrate a preferred embodiment of the present invention. But it will be apparent to those skilled in the art that alternative dimensions for the present invention may adequately address the issue of providing an apparatus for transporting a portable oxygen concentrator or other medical device, and all such alternative dimensions for the present invention are intended to be included in this specification.

While the present invention has been illustrated and described herein in terms of a preferred embodiment and several alternatives, it is to be understood that the apparatus described herein can have a multitude of additional uses and applications. Accordingly, the invention should not be limited to just the particular description and various drawing figures contained in this specification, that merely illustrate a preferred embodiment and application of the principles of the invention. It should be noted that terms of orientation and direction such as top, bottom, front, rear, etc. as used herein are used to distinguish elements from one another within exemplary embodiments and should therefore not be taken as limiting the scope of the present invention to any specific orientation. Moreover, the use of the terms first, second, etc. do not denote any order or importance, but rather are used to distinguish one element from another. Furthermore, the use of terms a, an, etc. do not denote a limitation of quantity, but rather denote the presence of at least one of the referenced item. Therefore, the detailed description and accompanying drawings are not to be taken in a limiting sense, but rather to enable the present invention.

What is claimed is:

1. A case for transporting a POC device, comprising:
a case body having one or more mesh panels to provide ventilation, wherein the case body is sized to receive the POC device, the one or more mesh panels are positioned to correspond to one or more functional sections of the POC that require ventilation, and wherein the case body forms a plurality of pass through openings for receiving one or more POC device connective apparatus;
one or more sets of wheels for supporting the case body and the POC device and for facilitating manual rolling of the case body and the POC device; and
a handle assembly that is retractable and telescopic.

2. The case for transporting a POC device as recited in claim 1, wherein the one or more POC device connective apparatus includes a power cable for connecting the POC to a power source and an oxygen hose for transporting oxygen from the POC to a patient.

3. The case for transporting a POC device as recited in claim 1, wherein the one or more sets of wheels includes four multi-directional spinning wheels.

4. The case for transporting a POC device as recited in claim 1, wherein the one or more sets of wheels and the handle assembly are sufficiently sturdy to provide mobility assistance to a patient.

5. The case for transporting a POC device as recited in claim 1 having an overall weight that allows an oxygen-deficient patent to transport the POC device without the assistance of a caretaker.

6. The case for transporting a POC device as recited in claim 1, wherein the case body includes an expandable top for expanding an interior size of the case body to accommodate a POC having a larger size.

7. The case for transporting a POC device as recited in claim 1, wherein one or more of the pass through openings are formed with double zipper fasteners for the ad hock creation of one or more additional pass through openings.

8. The case for transporting a POC device as recited in claim 1, wherein one or more of the pass through openings are flap-covered apertures.

9. The case for transporting a POC device as recited in claim 1, wherein one or more of the pass through openings are rubber ear-phone holes.

10. The case for transporting a POC device as recited in claim 1, wherein the handle apparatus includes one or more hooks for organizing one or more POC device connective apparatus.

11. A wheeled apparatus for transporting a medical device, comprising:
a case body having one or more ventilation panels to provide ventilation, wherein the case body is sized to receive the medical device, the one or more ventilation panels are positioned for the medical device, and wherein the case body forms a plurality of pass through openings for receiving one or more medical device connective apparatus;
one or more sets of wheels for supporting the case body and the medical device and for facilitating manual rolling of the case body and the medical device; and
a handle assembly that is retractable and telescopic and that provides maneuverability and mobility assistance to a user.

12. The case for transporting a medical device as recited in claim 11, wherein the one or more medical device connective apparatus includes a power cable for connecting the medical device to a power source.

13. The case for transporting a medical device as recited in claim 11, wherein the one or more sets of wheels includes four multi-directional spinning wheels.

14. The case for transporting a medical device as recited in claim 11, wherein the one or more sets of wheels and the handle assembly are sufficiently sturdy to provide mobility assistance to a patient.

15. The case for transporting a medical device as recited hi claim 11 having an overall weight that allows an oxygen-deficient patent to transport the medical device without the assistance of a caretaker.

16. The case for transporting a medical device as recited in claim 11, wherein the case body includes an expandable top for expanding an interior size of the case body to accommodate a medical device having a larger size.

17. The case for transporting a medical device as recited hi claim 11, wherein one or more of the pass through openings are formed with double zipper fasteners for the ad hock creation of one or more additional pass through openings.

18. The case for transporting a medical device as recited hi claim 11, wherein one or more of the pass through openings are flap-covered apertures.

19. The case for transporting a medical device as recited in claim 11, wherein one or more of the pass through openings are rubber ear-phone holes.

20. The case for transporting a medical device as recited in claim 11, wherein the handle apparatus includes one or more hooks for organizing one or more medical device connective apparatus.
